(19) United States
(12) Patent Application Publication
(10) Pub. No.: US 2025/0034507 A1
(43) Pub. Date: Jan. 30, 2025

(54) LIGHT SUPPLEMENTATION AND THERMAL INSULATION DEVICE FOR MICROALGAE PIPELINE CULTIVATION AND METHOD FOR MICROALGAE PIPELINE CULTIVATION

(71) Applicant: Algae Origin Inc., Newton, MA (US)

(72) Inventor: Bingchun CHANG, Newton, MA (US)

(73) Assignee: Algae Origin Inc., Newton, MA (US)

(21) Appl. No.: 18/226,843

(22) Filed: Jul. 27, 2023

(51) Int. Cl. F21V 9/30; C12M 1/00

(52) U.S. Cl. CPC C12M 41/08; C12M 31/02

(57) ABSTRACT

A light supplementation and thermal insulation device for microalgae pipeline cultivation and a method for microalgae pipeline cultivation is provided. The light supplementation and thermal insulation device has a combined light conversion film body, fixing parts located at an upper end and a lower end of the combined light conversion film body, and lifting systems; the combined light conversion film body comprises a bottom opaque film, a middle reflective film, and an upper light conversion film; the bottom opaque film, the middle reflective film, and the upper light conversion film are fixed together as a whole by the fixing parts at the upper end and the lower end; the lifting systems are connected to the fixing parts at the upper end. It solves the problem of difficult light supplementation during the daytime in microalgae pipeline cultivation and also addresses the issue of thermal insulation during the nighttime.

### TECHNICAL FIELD

The present invention relates to the technical field of microalgae cultivation, and in particular to a light supplementation and thermal insulation device for microalgae pipeline cultivation and a method for microalgae pipeline cultivation.

### BACKGROUND

Microalgae are unicellular aquatic organisms that propagate by self-division. They are widely distributed in both terrestrial and marine environments. The intensity of photosynthesis and the rate of biomass accumulation of microalgae far exceed those of terrestrial plants. Microalgae cell metabolism produces numerous active substances, including polysaccharides, proteins, and pigments. Therefore, microalgae have great prospects for exploitation.

Currently, the microalgae industry has become a strategic emerging industry. The existing microalgae cultivation modes mainly involve open raceway ponds, photobioreactors, and indoor closed systems with artificial light. The light source for microalgae photosynthesis is either solar energy or artificial LED light energy. Among them, open raceway ponds and closed photobioreactors are the mainstream cultivation modes, mainly utilizing sunlight as the light source for microalgae growth through photosynthesis. However, solar energy is unstable due to weather, season, and geographical factors, making it unable to meet the high requirements for microalgae nutrient accumulation and unit yield. The use of LED light can supplement the light energy, but it consumes a large amount of electricity, resulting in high cultivation costs, which is not conducive to energy conservation and emission reduction. Moreover, it is difficult to achieve large-scale applications.

### SUMMARY

In view of this, the present invention aims to provide a light supplementation and thermal insulation device for microalgae pipeline cultivation and a method for microalgae pipeline cultivation.

The present invention provides a light supplementation and thermal insulation device for microalgae pipeline cultivation, comprising a combined light conversion film body, fixing parts located at an upper end and a lower end of the combined light conversion film body, and lifting systems,
wherein the combined light conversion film body comprises a bottom opaque film, a middle reflective film, and an upper light conversion film;
the bottom opaque film, the middle reflective film, and the upper light conversion film are fixed together as a whole by the fixing parts at the upper end and the lower end;
the lifting systems are connected to the fixing parts at the upper end.

Preferably, the upper light conversion film comprises fluorescent powder, and the fluorescent powder is fluorescent powder capable of converting white light from sunlight into red light.

Preferably, the lifting systems comprise a first lifting system and a second lifting system; the first lifting system is used to control a height of an upper end of the combined light conversion film body from the ground and an inclination angle of the combined light conversion film body relative to the ground; the second lifting system is used to control the combined light conversion film to wrap a microalgae pipeline.

Preferably, the first lifting system comprises clamp strap hangers, suspension ropes, and control parts with wire ropes of the first lifting system; the clamp strap hangers are connected to the fixing parts at the upper end; one end of the suspension ropes is connected to the clamp strap hangers, and the other end is connected to the wire ropes of the first lifting system; the wire ropes of the first lifting system are connected to the control parts of the first lifting system.

Preferably, the second lifting system comprises clamp strap hangers shared with the first lifting system, suspension ropes shared with the first lifting system, and control parts with wire ropes of the second lifting system; the clamp strap hangers are connected to the fixing parts at the upper end; one end of the suspension ropes is connected to the clamp strap hangers, and the other end is connected to the wire ropes of the second lifting system; the wire ropes of the second lifting system are connected to the control parts of the second lifting system.

The present invention provides a method for microalgae pipeline cultivation using the light supplementation and thermal insulation device, comprising the following steps:
arranging the light supplementation and thermal insulation devices on both sides of a microalgae pipeline, with the upper light conversion film facing toward the microalgae pipeline;
during the daytime, controlling the height of the upper end of the combined light conversion film body from the ground to be 0 to 2.5 m, and controlling the inclination angle of the combined light conversion film body relative to a horizontal line to be 20 to 90°;
during the nighttime, controlling the height of the upper end of the combined light conversion film body from the ground to be 2 m, and wrapping the microalgae pipeline from both sides.

Preferably, during the daytime, the height of the upper end of the combined light conversion film body from the ground is adjusted to be 0.5 to 1.5 m based on the variation of an irradiation angle of sunlight.

Preferably, during the daytime, the inclination angle of the combined light conversion film body relative to the horizontal line is adjusted to be 30 to 60° based on the variation of an irradiation angle of sunlight.

Compared with the prior art, the present invention has the following beneficial effects: the light supplementation and thermal insulation device for microalgae pipeline cultivation provided by the present invention comprises a combined light conversion film body, fixing parts located at an upper end and a lower end of the combined light conversion film body, and lifting systems, wherein an upper light conversion film of the combined light conversion film body can effectively convert white light from sunlight into deep red light. After being irradiated by sunlight (visible light), the emission intensity of deep red light is enhanced by 25% to 35% and reflected onto the microalgae pipeline. This significantly contributes to the coloration of microalgae, an increase in OD value, and advancement in harvesting for microalgae pipeline cultivation. On the other hand, the light supplementation and thermal insulation device can cover both sides of the microalgae pipeline from top to bottom during the nighttime, providing effective thermal insulation.

Furthermore, the upper light conversion film used in the present invention is coated with aluminum-based non-rare earth oxide type inorganic fluorescent powder (Chinese Patent CN115261018A), which is relatively cost-effective.

The present invention features a simple process and equipment requirements, easy experimental conditions, a short production cycle, good reproducibility, wide adaptability, and low cost, and can meet the requirements of most microalgae pipeline cultivation. It solves the problem of difficult light supplementation during the daytime in microalgae pipeline cultivation and also addresses the issue of thermal insulation during the nighttime. It is suitable for large-scale industrial production.

### BRIEF DESCRIPTION OF THE DRAWINGS

FIG. 1 is a schematic view of the structure of the light supplementation and thermal insulation device for microalgae pipeline cultivation; and

FIG. 2 is a comparison diagram of optical density (OD) values during chlorella pipeline cultivation.

### DETAILED DESCRIPTION OF THE EMBODIMENTS

The present invention provides a light supplementation and thermal insulation device for microalgae pipeline cultivation, comprising a combined light conversion film body, fixing parts located at the upper end and the lower end of the combined light conversion film body, and lifting systems; the combined light conversion film body comprises a bottom opaque film, a middle reflective film, and an upper light conversion film; the bottom opaque film, the middle reflective film, and the upper light conversion film are fixed together as a whole by the fixing parts at the upper end and the lower end; the lifting systems are connected to the fixing parts at the upper end.

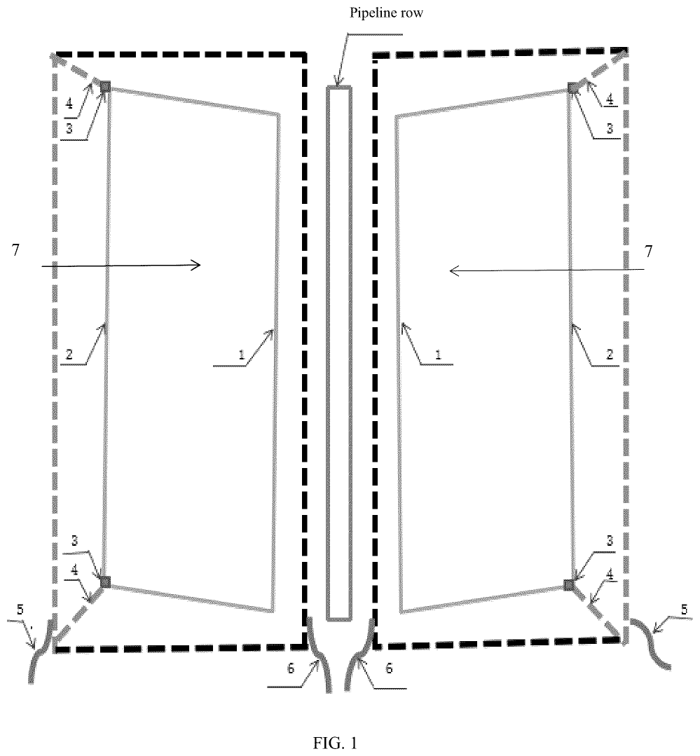

Referring to FIG. 1, 1 represents a fixing part such as a wood bar or aluminum bar at the lower end of the combined light conversion film, 2 represents a fixing part such as a wood bar or aluminum bar at the upper end of the combined light conversion film, 3 represents a clamp strap hanger, 4 represents two suspension ropes shared by the first lifting system and the second lifting system, 5 represents a control part with a wire rope in the first lifting system, 6 represents a control part with a wire rope in the second lifting system, and 7 represents a combined light conversion film.

In the present invention, the upper light conversion film comprises fluorescent powder, and the fluorescent powder is preferably fluorescent powder capable of converting white light from sunlight into red light; the fluorescent powder is further preferably the aluminum-based non-rare earth oxide type inorganic fluorescent powder disclosed in Chinese Patent CN115261018A. In the present invention, the bottom opaque film is preferably an aluminum film; the middle reflective film is preferably an advertising reflective film. In the present invention, the combined light conversion film body is obtained by combining the bottom opaque film, the middle reflective film, and the upper light conversion film in sequence. The shape of the combined light conversion film body is preferably a quadrilateral; the length and width of the combined light conversion film body are not particularly limited herein, which are determined based on the dimensions of the microalgae pipeline. In the specific implementation of the present invention, the specification of the combined light conversion film body is preferably (8-12) m*(2-3) m, further preferably (9-11) m*(2.2-2.8) m, and even more preferably 10 m*2.5 m.

In the present invention, the fixing parts 1, 2 are preferably located at the upper end and the lower end of the combined light conversion film body 7, and the fixing parts 1, 2 are used to fix the combined light conversion film body 7. In the present invention, each fixing part 1, 2 is preferably of a strip form, further preferably a wood bar or an aluminum bar.

In the present invention, the lifting systems preferably comprise a first lifting system and a second lifting system; the first lifting system is used to control the height of the upper end of the combined light conversion film body 7 from the ground and the inclination angle of the combined light conversion film body 7 relative to the ground; the first lifting system preferably comprises clamp strap hangers 3, suspension ropes 4, and control parts with wire ropes 5 of the first lifting system; the materials of the clamp strap hangers 3 and the suspension ropes 5 are not particularly limited herein, and conventional materials in the art can be used. In the specific implementation of the present invention, the clamp strap hangers 3 and suspension ropes are preferably made of stainless steel. In the present invention, the control part 5 of the first lifting system is an automatic or manual control element, preferably a manual control element; the control part 5 of the first lifting system preferably comes with a wire rope. In the present invention, the clamp strap hangers 3 are connected to the fixing parts 1 at the upper end; one end of the suspension ropes 4 is connected to the clamp strap hangers 3, and the other end is connected to the wire ropes of the first lifting system; the wire ropes of the first lifting system are connected to the control parts 5 of the first lifting system.

In the present invention, the number of the clamp strap hangers and the number of the suspension ropes are equal, preferably greater than or equal to 1, and further preferably 2 to 4.

In the present invention, the second lifting system is used to control the combined light conversion film 7 to wrap the microalgae pipeline. The second lifting system preferably comprises clamp strap hangers 3 shared with the first lifting system, suspension ropes 4 shared with the first lifting system, and control parts with wire ropes 6 of the second lifting system; in the present invention, the clamp strap hangers 3 are connected to the fixing parts 1 at the upper end; one end of the suspension ropes 4 is connected to the clamp strap hangers 3, and the other end is connected to the wire ropes of the second lifting system; the wire ropes of the second lifting system are connected to the control parts 6 of the second lifting system.

The present invention further provides a method for microalgae pipeline cultivation using the light supplementation and thermal insulation device, comprising the following steps: arranging the light supplementation and thermal insulation devices on both sides of a microalgae pipeline, with the upper light conversion film facing toward the microalgae pipeline; during the daytime, controlling the height of the upper end of the combined light conversion film body from the ground to be 0 to 2.5 m, and controlling the inclination angle of the combined light conversion film body relative to the horizontal line to be 20 to 90°; during the nighttime, controlling the height of the upper end of the film body from the ground to be 2.5 m, and allowing the combined light conversion film body to wrap the microalgae pipeline from both sides.

In the present invention, the horizontal distance between the light supplementation and thermal insulation device and the microalgae pipeline is preferably to be 0.5 to 1.5 m. During the daytime, the height of the upper end of the combined light conversion film body from the ground is preferably controlled to be 0.5 to 1.5 m, and the inclination angle of the combined light conversion film body relative to the horizontal line is preferably controlled to be 30 to 60°, further preferably 40 to 50°. During the daytime, the height and inclination angle of the combined light conversion film body relative to the ground can be adjusted according to the irradiation of sunlight, so as to obtain the strongest reflected red light. The specific condition and mode of the microalgae cultivation are not particularly limited herein, and the conventional conditions for the pipeline microalgae cultivation in the art can be used. The types of microalgae are not particularly limited herein, and any conventional microalgae types in the art may be used. In the specific implementation of the present invention, the microalgae are preferably chlorella, spirulina, or haematococcus pluvialis.

The technical solutions provided by the present invention will be described in detail below with reference to the examples, which, however, should not be construed as limiting the protection scope of the present invention.

The preparation method for the light supplementation and thermal insulation device for microalgae pipeline cultivation is as follows:
1) mixing a finished product of the aluminum-based non-rare earth oxide type inorganic fluorescent powder (Chinese Patent CN115261018A) with PDMS adhesive in a mass ratio of 1:9; blow molding the mixture into a light conversion film of 10 m*2.5 m; forming a combined light conversion film body of 10 m*2.5 m by adding an opaque film on the bottom layer, an advertising reflective film on the middle layer, and a light conversion film on the upper layer (the upper light conversion film must face the plant);
2) fixing the bottom opaque film, the middle reflective film, and the upper light conversion film together as a whole with fixing parts 1, 2 such as wood bars or aluminum bars at the upper end and the lower end; connecting the clamp strap hangers 3 to the fixing parts 1 such as wood bars or aluminum bars at the upper end; connecting one end of the suspension ropes 4 to the clamp strap hangers 3, and dividing the other end of the suspension ropes into two parts, with one part connected to the control parts 5 of the first lifting system through the wire ropes of the first lifting system and the other part connected to the control parts 6 of the second lifting system through the wire ropes of the second lifting system, the control parts of the second lifting system being installed in the middle of the microalgae pipeline cultivation row as shown in FIG. 1.

### Example 1

The light supplementation and thermal insulation device according to the present invention was installed on both sides of the chlorella pipeline, with the upper light conversion film facing the chlorella pipeline. During the daytime, the first lifting system was used to adjust the inclination angle between the surface of the combined light conversion film body and the horizontal plane to 30°, and the first lifting system manual controller was used to raise the upper end of the combined light conversion film body to a height of 0.5 m from the ground. This setup was applied to the chlorella pipeline cultivation 1 to cultivate chlorella and observe the growth process thereof. During the nighttime, the second lifting system manual controller was used to cover the left side and the right side of the chlorella pipeline cultivation row from top to bottom. The growth process was observed, and the liquid temperature was determined.

### Example 2

The light supplementation and thermal insulation device according to the present invention was installed on both sides of the chlorella pipeline, with the upper light conversion film facing the chlorella pipeline. During the daytime, the first lifting system was used to adjust the inclination angle between the surface of the combined light conversion film body and the horizontal plane to 30°, and the first lifting system manual controller was used to raise the upper end of the combined light conversion film body to a height of 0.9 m from the ground. This setup was applied to the chlorella pipeline cultivation 2 to cultivate chlorella and observe the growth process thereof. During the nighttime, the second lifting system manual controller was used to cover the left side and the right side of the chlorella pipeline cultivation row from top to bottom. The growth process was observed, and the liquid temperature was determined.

### Example 3

The light supplementation and thermal insulation device according to the present invention was installed on both sides of the chlorella pipeline, with the upper light conversion film facing the chlorella pipeline. During the daytime, the first automatic lifting system was used to adjust the inclination angle between the surface of the combined light conversion film body and the horizontal plane to 45°, and the first lifting system manual controller was used to raise the upper end of the combined light conversion film body to a height of 0.9 m from the ground. This setup was applied to the chlorella pipeline cultivation 3 to cultivate chlorella and observe the growth process thereof. During the nighttime, the second lifting system manual controller was used to cover the left side and the right side of the chlorella pipeline cultivation row from top to bottom. The growth process was observed, and the liquid temperature was determined.

### Example 4

The light supplementation and thermal insulation device according to the present invention was installed on both sides of the chlorella pipeline, with the upper light conversion film facing the chlorella pipeline. During the daytime, the first lifting system was used to adjust the inclination angle between the surface of the combined light conversion film body and the horizontal plane to 45°, and the automatic lifting system handle or controller was used to raise the film surface to a height of 1.3 m from the ground. This setup was applied to the chlorella pipeline cultivation 4 to cultivate chlorella and observe the growth process thereof. During the nighttime, the second lifting system manual controller was used to cover the left side and the right side of the chlorella pipeline cultivation row from top to bottom. The growth process was observed, and the liquid temperature was determined.

### Example 5

The light supplementation and thermal insulation device according to the present invention was installed on both sides of the chlorella pipeline, with the upper light conversion film facing the chlorella pipeline. During the daytime, the first lifting system was used to adjust the inclination angle between the surface of the combined light conversion film body and the horizontal plane to 60°, and the first lifting system manual controller was used to raise the upper end of the combined light conversion film body to a height of 1.3 m from the ground. This setup was applied to the chlorella pipeline cultivation 5 to cultivate chlorella and observe the growth process thereof. During the nighttime, the second lifting system manual controller was used to cover the left side and the right side of the chlorella pipeline cultivation row from top to bottom. The growth process was observed, and the liquid temperature was determined.

### Example 6

The light supplementation and thermal insulation device according to the present invention was installed on both sides of the chlorella pipeline, with the upper light conversion film facing the chlorella pipeline. During the daytime, the first lifting system was used to adjust the inclination angle between the surface of the combined light conversion film body and the horizontal plane to 60°, and the first lifting system manual controller was used to raise the upper end of the combined light conversion film body to a height of 1.7 m from the ground. This setup was applied to the chlorella pipeline cultivation 6 to cultivate chlorella and observe the growth process thereof. During the nighttime, the second lifting system manual controller was used to cover the left side and the right side of the chlorella pipeline cultivation row from top to bottom. The growth process was observed, and the liquid temperature was determined.

### Example 7

The light supplementation and thermal insulation device according to the present invention was installed on both sides of the chlorella pipeline, with the upper light conversion film facing the chlorella pipeline. At 8:00 during the daytime, the first lifting system was used to adjust the inclination angle between the surface of the combined light conversion film body and the horizontal plane to 30°, and to raise the upper end of the combined light conversion film body to a height of 0.8 m from the ground. At 11:00, the first lifting system was used to adjust the inclination angle between the surface of the combined light conversion film body and the horizontal plane to 45° and to raise the upper end of the combined light conversion film body to a height of 1.2 m from the ground. At 15:00, the first lifting system was used to adjust the inclination angle between the surface of the combined light conversion film body and the horizontal plane to 60°, and to raise the upper end of the combined light conversion film body to a height of 1.5 m from the ground. This setup was applied to the chlorella pipeline cultivation 7 to cultivate chlorella and observe the growth process thereof. During the nighttime, the second lifting system manual controller was used to cover the left side and the right side of the chlorella pipeline cultivation row from top to bottom. The growth process was observed, and the liquid temperature was determined.

### Comparative Example 8

A chlorella pipeline cultivation in the same room without the use of the light supplementation and thermal insulation device according to the present invention was taken as a control sample. LED plant illuminating lights (a total of 20 lights installed on both sides of the 10-meter-long pipeline, with 1.2 m per light, 18 W per light) were installed on both sides of the chlorella pipeline. The growth process of chlorella pipeline cultivation 7 was observed.

### Comparative Example 9

A chlorella pipeline cultivation in the same room without the use of the light supplementation and thermal insulation device according to the present invention was taken as a control sample. LED plant lights were not installed. The growth process of chlorella pipeline cultivation 8 was observed.

### Experimental Results

After 7 days of cultivation, the OD values of the chlorella in each chlorella pipeline are shown in Table 1.

TABLE 1
Comparison of OD values during chlorella pipeline cultivation

| | | Days | | | | | | | |
|---|---|---|---|---|---|---|---|---|---|
| OD value | Groups | 1 | 2 | 3 | 4 | 5 | 6 | 7 | 8 |
| | Example 1 | 0.695 | 1.071 | 1.302 | 1.603 | 1.803 | 2.224 | 2.341 | 2.645 |
| | Example 2 | 0.696 | 1.106 | 1.354 | 1.672 | 1.901 | 2.328 | 2.415 | 2.851 |
| | Example 3 | 0.698 | 1.112 | 1.361 | 1.681 | 1.902 | 2.342 | 2.435 | 2.873 |
| | Example 4 | 0.701 | 1.121 | 1.375 | 1.702 | 1.925 | 2.371 | 2.461 | 2.893 |
| | Example 5 | 0.705 | 1.132 | 1.392 | 1.721 | 2.001 | 2.402 | 2.508 | 2.908 |
| | Example 6 | 0.709 | 1.143 | 1.401 | 1.732 | 2.023 | 2.435 | 2.524 | 2.935 |
| | Example 7 | 0.723 | 1.171 | 1.422 | 1.751 | 2.136 | 2.545 | 2.883 | 3.234 |
| | Comparative | 0.693 | 1.041 | 1.261 | 1.549 | 1.736 | 2.143 | 2.305 | 2.538 |
| | Example 8 | | | | | | | | |
| | Comparative | 0.691 | 0.968 | 1.133 | 1.377 | 1.521 | 1.886 | 2.108 | 2.293 |
| | Example 9 | | | | | | | | |

The comparison of one harvest cycle during chlorella pipeline cultivation is shown in Table 2.

TABLE 2
Comparison table of one harvest cycle
during chlorella pipeline cultivation

One harvest cycle

| Groups | Hour(s) | Effective days |
|---|---|---|
| Example 1 | 133 | 5.5 |
| Example 2 | 130 | 5.4 |
| Example 3 | 126 | 5.3 |
| Example 4 | 124 | 5.2 |
| Example 5 | 122 | 5.1 |
| Example 6 | 120 | 5.0 |
| Example 7 | 110 | 4.6 |
| Comparative Example 8 | 140 | 5.8 |
| Comparative Example 9 | 168 | 7.0 |

The comparison of the liquid temperatures in 7 days during chlorella pipeline cultivation is shown in Table 3.

TABLE 3
Comparison of liquid temperatures in 7 days during chlorella pipeline cultivation

| | Monday | Tuesday | Wednesday | Thursday | Friday | Saturday | Sunday |
|---|---|---|---|---|---|---|---|
| Nighttime | (° C.) | (° C.) | (° C.) | (° C.) | (° C.) | (° C.) | (° C.) |
| Example 1 | 19.1 | 17.5 | 18.3 | 19.4 | 19.8 | 19.5 | 18.7 |
| Example 2 | 19.2 | 17.3 | 18.3 | 19.3 | 19.7 | 19.6 | 18.6 |
| Example 3 | 19.3 | 17.2 | 18.3 | 19.2 | 19.6 | 19.5 | 18.9 |
| Example 4 | 19.1 | 17.3 | 18.4 | 19.1 | 19.7 | 19.3 | 18.7 |
| Example 5 | 19.2 | 17.4 | 18.3 | 19.2 | 19.8 | 19.4 | 18.8 |
| Example 6 | 19.1 | 17.2 | 18.5 | 19.3 | 19.9 | 19.6 | 18.7 |
| Example 7 | 19.2 | 17.3 | 18.4 | 19.4 | 19.7 | 19.5 | 18.8 |
| Comparative Example 8 | 15.3 | 13.4 | 14.3 | 15.3 | 15.6 | 15.5 | 14.3 |
| Comparative Example 9 | 14.5 | 12.2 | 13.1 | 13.3 | 14.7 | 14.3 | 13.2 |

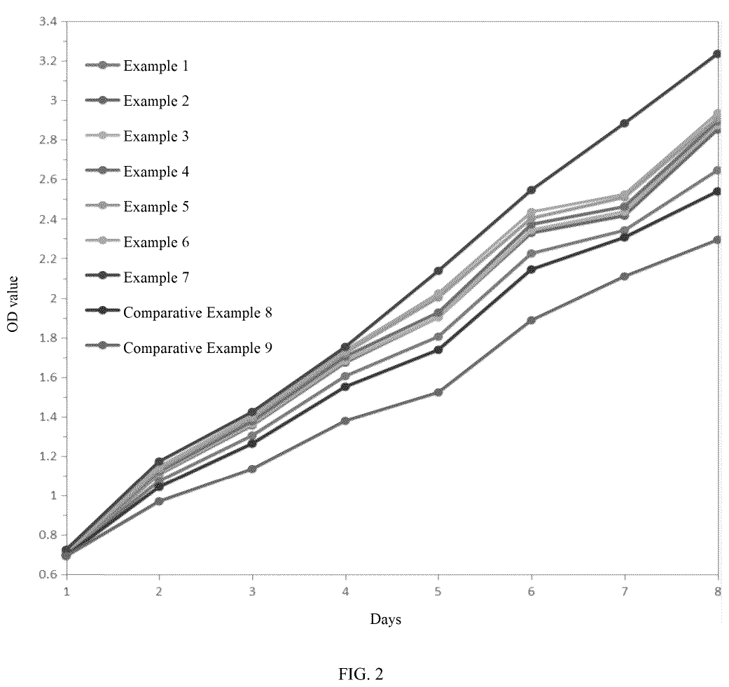

According to the methods in Examples 1, 2, 3, 4, 5, 6, and 7, the light supplementation and thermal insulation device was applied respectively to the chlorella pipeline cultivation 1, 2, 3, 4, 5, 6, and 7 for their growth, which were compared with chlorella 8 (with LED plant lights installed) and chlorella 9 without the use of the light conversion film light supplementation device. As shown in Table 1 and FIG. 2, the OD values for the growth of chlorella pipeline 1, 2, 3, 4, 5, 6, and 7 were higher than the OD values for the growth of chlorella pipeline 8, and the OD values for the growth of chlorella pipeline 1, 2, 3, 4, 5, 6, 7, and 8 were significantly higher than the OD values for the growth of chlorella pipeline 9. This indicates that the light supplementation and thermal insulation device provided by the present invention can increase the OD values for chlorella growth, outperforming the LED plant lights.

According to the methods in Examples 1, 2, 3, 4, 5, 6, and 7, the light supplementation and thermal insulation device was applied respectively to the chlorella pipeline cultivation 1, 2, 3, 4, 5, 6, and 7 for their growth. As shown in Table 1 and FIG. 2, the OD values for the growth of chlorella 2 were larger than the OD values for the growth of chlorella 1, the OD values for the growth of chlorella 4 were larger than the OD values for the growth of chlorella 3, the OD values for the growth of chlorella 6 were larger than the OD values for the growth of chlorella 5, and the OD values for the growth of chlorella 7 were larger than the OD values for the growth of chlorella 1, 2, 3, 4, 5, and 6. This was because the height of the upper end of the combined light conversion film body in Examples 2, 4, 6, and 7 from the ground was adjusted, enabling better adaptation to the movement of sunlight and better reflection of sunlight.

According to the methods in Examples 1, 2, 3, 4, 5, 6, and 7, the light supplementation and thermal insulation device was applied respectively to the chlorella pipeline cultivation 1, 2, 3, 4, 5, 6, and 7 for their growth. As shown in Table 1 and FIG. 2, the OD values for the growth of chlorella 3 were larger than the OD values for the growth of chlorella 2, the OD values for the growth of chlorella 5 were larger than the OD values for the growth of chlorella 4, and the OD values for the growth of chlorella 7 were larger than the OD values for the growth of chlorella 1, 2, 3, 4, 5, and 6. This was because the inclination angle of the combined light conversion film body in Examples 3, 5, and 7 were changed, enabling better adaptation to the movement of sunlight and better reflection of sunlight.

According to the methods in Examples 1, 2, 3, 4, 5, 6, and 7, the light supplementation and thermal insulation device was applied respectively to the chlorella pipeline cultivation 1, 2, 3, 4, 5, 6, and 7 for their growth, which were compared with pipeline-cultivated chlorella 8 (with LED plant lights installed) and chlorella 9 (with no LED plant lights installed) without the use of the light conversion film light supplementation and thermal insulation device. Taking an inoculum concentration of 0.5 and a mature harvesting concentration of 2 as an example, Table 2 shows that within one harvest cycle, the growing days of chlorella 1, 2, 3, 4, 5, 6, 7, and 8 were shorter than the growing days of chlorella 9, and the growing days of chlorella 1, 2, 3, 4, 5, 6, and 7 were shorter than the growing days of chlorella 8 (with LED plant lights installed). This indicates that the light supplementation and thermal insulation device according to the present invention can promote the early harvesting of chlorella, outperforming the LED plant lights.

According to the methods in Examples 1, 2, 3, 4, 5, 6, and 7, the light supplementation and thermal insulation device was applied respectively to the chlorella pipeline cultivation 1, 2, 3, 4, 5, 6, and 7 for their growth, which were compared with chlorella 8 (with LED plant lights installed) and chlorella 9 (with no LED plant lights installed) without the use of the light conversion film light supplementation and thermal insulation device. As shown in Table 3, during each night of a week, the liquid temperatures of chlorella 1, 2, 3, 4, 5, 6, and 7 were 5° C. higher than the liquid temperatures of chlorella 9 (with no LED plant lights installed), and the liquid temperatures of chlorella 1, 2, 3, 4, 5, 6, and 7 were 4° C. higher than the liquid temperatures of chlorella 8 (with LED plant lights installed). This indicates that the light supplementation and thermal insulation device provided by the present invention can provide thermal insulation, outperforming the LED plant lights.

Based on the above examples, the light supplementation and thermal insulation device provided by the present invention can adjust the inclination angles of the left and right sides and the height from the ground according to the movement of sunlight. It exhibits superior effects in terms of the OD value for the growth of chlorella and early harvesting compared with the use of LED plant lights, promoting the rapid growth of microalgae pipeline cultivation. At the same time, it can increase the liquid temperature for microalgae cultivation by 5° C., providing thermal insulation and promoting the rapid growth of microalgae pipeline cultivation in an environment of 15° C. to 20° C.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A light supplementation and thermal insulation device for microalgae pipeline cultivation, comprising a combined light conversion film body, a fixing part located at an upper end and a fixing part located at a lower end of the combined light conversion film body, and lifting systems,
wherein the combined light conversion film body comprises a bottom opaque film, a middle reflective film, and an upper light conversion film;
the bottom opaque film, the middle reflective film, and the upper light conversion film are fixed together as a whole by the fixing part at the upper end and the fixing part at the lower end;
the lifting systems are connected to the fixing part at the upper end.

2. The light supplementation and thermal insulation device according to claim 1, wherein the upper light conversion film comprises fluorescent powder, and the fluorescent powder is fluorescent powder capable of converting white light from sunlight into red light.

3. The light supplementation and thermal insulation device according to claim 1, wherein the lifting systems comprise a first lifting system and a second lifting system; the first lifting system is used to control a height of the upper end of the combined light conversion film body from the ground and an inclination angle of the combined light conversion film body relative to the ground; the second lifting system is used to control the combined light conversion film to wrap a microalgae pipeline.

4. The light supplementation and thermal insulation device according to claim 3, wherein the first lifting system comprises clamp strap hangers, suspension ropes, and control parts with wire ropes of the first lifting system; the clamp strap hangers are connected to the fixing part at the upper end; one end of the suspension ropes is connected to the clamp strap hangers, and the other end is connected to the wire ropes of the first lifting system; the wire ropes of the first lifting system are connected to the control parts of the first lifting system.

5. The light supplementation and thermal insulation device according to claim 3, wherein the second lifting system comprises clamp strap hangers shared with the first lifting system, suspension ropes shared with the first lifting system, and control parts with wire ropes of the second lifting system; the clamp strap hangers are connected to the fixing part at the upper end; one end of the suspension ropes is connected to the clamp strap hangers, and the other end is connected to the wire ropes of the second lifting system; the wire ropes of the second lifting system are connected to the control parts of the second lifting system.

6. A method for microalgae pipeline cultivation using the light supplementation and thermal insulation device according to claim 1, comprising the following steps:
arranging the light supplementation and thermal insulation devices on both sides of a microalgae pipeline, with the upper light conversion film facing toward the microalgae pipeline;
during the daytime, controlling a height of the upper end of the combined light conversion film body from the ground to be 0.5 to 2.5 m, and controlling an inclination angle of the combined light conversion film body relative to a horizontal line to be 20 to 90°;
during the nighttime, controlling the height of the upper end of the combined light conversion film body from the ground to be 2.5 m, and wrapping the microalgae pipeline from both sides.

7. The method according to claim 6, wherein during the daytime, the height of the upper end of the combined light conversion film body from the ground is adjusted to be 0.5 m to 1.5 m based on the variation of an irradiation angle of sunlight.

8. The method according to claim 6, wherein during the daytime, the inclination angle of the combined light conversion film body relative to the horizontal line is adjusted to be 30° to 60° based on the variation of an irradiation angle of sunlight.

9. The light supplementation and thermal insulation device according to claim 2, wherein the lifting systems comprise a first lifting system and a second lifting system; the first lifting system is used to control a height of the upper end of the combined light conversion film body from the ground and an inclination angle of the combined light conversion film body relative to the ground; the second lifting system is used to control the combined light conversion film to wrap a microalgae pipeline.

10. The light supplementation and thermal insulation device according to claim 9, wherein the first lifting system comprises clamp strap hangers, suspension ropes, and control parts with wire ropes of the first lifting system; the clamp strap hangers are connected to the fixing part at the upper end; one end of the suspension ropes is connected to the clamp strap hangers, and the other end is connected to the wire ropes of the first lifting system; the wire ropes of the first lifting system are connected to the control parts of the first lifting system.

11. The light supplementation and thermal insulation device according to claim 9, wherein the second lifting system comprises clamp strap hangers shared with the first lifting system, suspension ropes shared with the first lifting system, and control parts with wire ropes of the second lifting system; the clamp strap hangers are connected to the fixing part at the upper end; one end of the suspension ropes is connected to the clamp strap hangers, and the other end is connected to the wire ropes of the second lifting system; the wire ropes of the second lifting system are connected to the control parts of the second lifting system.

12. The method according to claim 6, wherein the upper light conversion film comprises fluorescent powder, and the fluorescent powder is fluorescent powder capable of converting white light from sunlight into red light.

13. The method according to claim 6, wherein the lifting systems comprise a first lifting system and a second lifting system; the first lifting system is used to control the height of the upper end of the combined light conversion film body from the ground and the inclination angle of the combined light conversion film body relative to the ground; the second lifting system is used to control the combined light conversion film to wrap the microalgae pipeline.

14. The method according to claim 13, wherein the first lifting system comprises clamp strap hangers, suspension ropes, and control parts with wire ropes of the first lifting system; the clamp strap hangers are connected to the fixing part at the upper end; one end of the suspension ropes is connected to the clamp strap hangers, and the other end is connected to the wire ropes of the first lifting system; the wire ropes of the first lifting system are connected to the control parts of the first lifting system.

15. The method according to claim 13, wherein the second lifting system comprises clamp strap hangers shared with the first lifting system, suspension ropes shared with the first lifting system, and control parts with wire ropes of the second lifting system; the clamp strap hangers are connected to the fixing part at the upper end; one end of the suspension ropes is connected to the clamp strap hangers, and the other end is connected to the wire ropes of the second lifting system; the wire ropes of the second lifting system are connected to the control parts of the second lifting system.

16. The method according to claim 12, wherein the lifting systems comprise a first lifting system and a second lifting system; the first lifting system is used to control the height of the upper end of the combined light conversion film body from the ground and the inclination angle of the combined light conversion film body relative to the ground; the second lifting system is used to control the combined light conversion film to wrap a microalgae pipeline.

17. The method according to claim 16, wherein the first lifting system comprises clamp strap hangers, suspension ropes, and control parts with wire ropes of the first lifting system; the clamp strap hangers are connected to the fixing part at the upper end; one end of the suspension ropes is connected to the clamp strap hangers, and the other end is connected to the wire ropes of the first lifting system; the wire ropes of the first lifting system are connected to the control parts of the first lifting system.

18. The method according to claim 16, wherein the second lifting system comprises clamp strap hangers shared with the first lifting system, suspension ropes shared with the first lifting system, and control parts with wire ropes of the second lifting system; the clamp strap hangers are connected to the fixing part at the upper end; one end of the suspension ropes is connected to the clamp strap hangers, and the other end is connected to the wire ropes of the second lifting system; the wire ropes of the second lifting system are connected to the control parts of the second lifting system.

19. The method according to claim 12, wherein during the daytime, the height of the upper end of the combined light conversion film body from the ground is adjusted to be 0.5 m to 1.5 m based on the variation of an irradiation angle of sunlight.

20. The method according to claim 12, wherein during the daytime, the inclination angle of the combined light conversion film body relative to the horizontal line is adjusted to be 30° to 60° based on the variation of an irradiation angle of sunlight.
